(19) United States
(12) Patent Application Publication
(10) Pub. No.: US 2026/0198876 A1
(43) Pub. Date: Jul. 16, 2026

(54) APPARATUS AND METHOD FOR REGISTRATION OF A SHAPE-DETECTING INSTRUMENT

(71) Applicant: Siemens Healthineers AG, Forchheim (DE)

(72) Inventor: Marcus Pfister, Forchheim (DE)

(21) Appl. No.: 19/449,028

(22) Filed: Jan. 14, 2026

(30) Foreign Application Priority Data
Jan. 14, 2025 (DE) 10 2025 101 121.4

(51) Int. Cl. A61B 6/12; A61B 6/04; A61B 34/20; G06T 7/70

(52) U.S. Cl. CPC A61B 6/12; A61B 6/0407; G06T 7/70; A61B 2034/2061

(57) ABSTRACT

An apparatus and a method for reciprocal registration of a shape-detecting instrument, an imaging X-ray system, and an X-ray system including a corresponding apparatus are provided. The apparatus includes a registration facility. The registration facility is configured such that the shape-detecting instrument, until attaining a registering position, may be introduced into the registration facility. The spatial relative position of the registering position may be kept at least temporarily unchanged in relation to the X-ray system and is assumed to be known for the method. A detecting facility that is configured to detect when the shape-detecting instrument attains the registering position is also provided. A registering signal may be generated by the detecting facility when the detecting facility detects that the shape-detecting instrument attains the registering position.

This application claims the benefit of German Patent Application No. DE 10 2025 101 121.4, filed on Jan. 14, 2025, which is hereby incorporated by reference in its entirety.

### BACKGROUND

The present embodiments relate to an apparatus and to a method for reciprocal registration of a shape-detecting instrument, and an imaging X-ray system as well as to an X-ray system including a corresponding apparatus.

In the case of minimally invasive interventions using X-ray imaging (e.g., with C-Arm angiography systems), therapies and diagnoses are carried out with instruments that are introduced into the body through small incisions (e.g., in the groin). X-ray imaging is used for navigation to an intracorporeal or intravascular target. Catheters and other instruments as well as anatomical features may be visualized with the aid of X-ray imaging. However, X-ray imaging has the drawback that the patient, as well as the medical staff, is exposed to a dose of radiation.

To overcome the drawback of the dose of radiation, it is known to use shape-detecting instruments that enable detection of the peculiar shape or position without the use of X-ray radiation. The shape may be detected, for example, via fiber-optic 3D-shape sensing based on light conduction. The modulation of the light conduction in the optical fiber is used, for example, for determining the shape. It is known to use this technology, for example, for the visualization of guide wires. The shape is detected without X-ray imaging and thereby without a dose of X-ray radiation. With knowledge of the position of the starting point of the optical fiber, it is possible to also derive the spatial position in the fiber from the detected spatial shape. Further, X-ray radiation-free approaches are known, for example, based on electromagnetic tracking in which electromagnetic fields are used to determine the spatial position. The spatial shape of the tracked instrument may also be derived from the detected position(s). Hereinafter, the term “shape-detecting” may to be that a spatial shape or position of an instrument may be detected without the use of X-ray radiation.

Systems for determining shape and position may use their own spatial coordinate system. In fiber-optic 3D-shape sensing, it is known, for example, to anchor the coordinate system of the fiber-optic system close to the relevant target location of the position determination and in a suitable alignment with the target location. X-ray systems may also use their own spatial coordinate system. As a rule, the coordinate system of an X-ray system is anchored in relation to X-ray emitters or X-ray detectors. The items of shape and position information of X-ray radiation-free shape-detecting systems (e.g., fiber-optic systems) may be able to be used in the course of procedures with X-ray imaging. For this purpose, the definition of a shared coordinate system of the shape-detecting system and of the X-ray system is necessary, or the definition of a spatial transformation rule between the two coordinate systems. With such a transformation rule, reference is also made to the registration of the two coordinate systems or to the registration of the X-ray system and the shape-detecting instrument. Hereinafter, the terms “transformation” and “registration” may refer to a spatial transformation or spatial registration. Hereinafter, the term “registration” may be that a transformation rule is ascertained between coordinate systems, a coordinate system is transformed into another coordinate system, or coordinate systems are transformed into a shared, single coordinate system.

The registration of the coordinate systems of an X-ray system and a shape-detecting instrument is basically known in the prior art. As a rule, two X-ray images of the shape-detecting instrument are recorded for registration of the two coordinate systems. The position of the instrument in relation to the X-ray system is reconstructed based on the two X-ray images. The reconstructed position is then registered with the item of position information of the shape-detecting instrument. This method requires the recording of two or more X-ray images and is thereby laborious and time-consuming. The accuracy of this method depends on the accuracy with which the position of the instrument may be reconstructed from the X-ray images.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the known methods may be improved by an apparatus, a method, and an X-ray system including the apparatus.

The present embodiments include an apparatus and a method that are based on a registration facility that is fixed and shape-stable in relation to an X-ray system. The spatial relative position of the registration facility in relation to the X-ray system and the shape are known from the outset and are used as the basis for the registration.

The present embodiments thereby discloses a fast, precise, uncomplicated, and radiation-free method for the registration of the coordinate systems of an X-ray system and a shape-detecting instrument.

The apparatus of the present embodiments includes a registration facility (e.g., a registration feature) configured such that the shape-detecting instrument, until attaining a registering position, may be introduced into the registration facility. The spatial relative position of the registering position in relation to the X-ray system is known from the outset. The apparatus also includes a detecting facility (e.g., a detection device including a sensor) configured to detect when the shape-detecting instrument attains the registering position. A registering signal may be generated by the detecting facility when the detecting facility detects that the shape-detecting instrument attains the registering position.

With respect to the apparatus of the present embodiments, the apparatus has two fundamental components, for example, a registration facility and a detecting facility. The registration facility is formed such that the instrument may be introduced at least up to a certain position, the registering position. This position in relation to the X-ray system is assumed to be known; in other words, the position is therefore registered with the X-ray system. This may either be known from the outset owing to knowledge of the construction, or may have been ascertained in advance by preceding X-rays by the X-ray system. The spatial relative position of the registering position in relation to the X-ray system is to be kept unchanged, at least temporarily, for carrying out the reciprocal registration, and thereby, may be assumed to be known from the outset and does not have to be ascertained or reconstructed specifically for carrying out the registration. The detecting facility recognizes when the instrument has attained the registering position, and generates a signal in order to indicate this.

So the registering position may be kept at least temporarily unchanged in relation to the X-ray system, the apparatus may be permanently integrated from the outset (e.g., in a component of the imaging X-ray facility; in the X-ray system or the patient table). Alternatively, it may be provided that the apparatus may be temporarily permanently connected to one of the components of the imaging facility and then may be detached from the one component again. For example, the apparatus may be placed on the patient table or be mounted or pushed onto the patient table.

The registration facility is an apparatus or apparatus component that brings the instrument into the registering position. The detecting facility is a facility that recognizes when the instrument has attained the registering position, and then generates a signal. The detecting facility may be configured, for example, as a contact sensor or touch sensor, or as a capacitive or optical sensor. These may also be conventional and available sensors that are uncomplicated, reliable, and inexpensive.

The registering position is the position that the instrument is to adopt in the registration facility in order to be correctly registered. The spatial relative position of the registering position in relation to the X-ray system is known from the outset. The spatial relative position may, for example, be strictly predefined in terms of construction. The registering signal is generated by the detecting facility when the instrument has attained the registering position. The reciprocal registration of instrument and X-ray system may be ascertained by a computer-implemented registration method in response to the generation of the registering signal.

The registration method may be carried out by the X-ray system or by the shape-detecting instrument because the X-ray system and the shape-detecting instrument each have a coordinate system. The X-ray system includes a control facility (e.g., a controller), and the shape-detecting instrument includes a monitoring facility. One of the two facilities or also both may carry out the registration method. The registering signal is therefore transmitted by the detecting facility either to the control facility of the X-ray system or to the monitoring facility of the shape-detecting instrument. As soon as the registering signal is received, the registration method is started in the receiving facility.

The present embodiments may reduce the effort and time requirement for the registration of the coordinate systems since no additional X-ray images are to be recorded. The present embodiments may improve the precision of the registration since with the previously known registering position, the exact relative position of the instrument in relation to the X-ray system is known. The present embodiments may reduce the effort since no additional steps, such as recording X-ray images, are required, and since the registration method may be automatically triggered based on the registering signal. The present embodiments may reduce the exposure to radiation for patients and medical staff since no additional X-ray images are to be recorded for the purpose of registration.

A development of the present embodiments discloses an apparatus in which the registration facility is formed such that when introduced into the registration facility, the shape-detecting instrument is deformed into a shape similar to the shape of the introduction opening of a registration facility. The introduction opening is formed such that the introduction opening is able to receive the relevant shape-detecting instrument (e.g., longitudinally and in a tube-like manner for an elongate tubular instrument). Similarly, this provides that the shape of the shape-detecting instrument, when the shape-detecting instrument is introduced in the registration facility, approximately corresponds to the shape of the introduction opening of the registration facility (e.g., is approximately congruent or in its course approximately conforms with the introduction opening). The accuracy of the similarity or conformity depends substantially on the mechanical play between the shape-detecting instrument and the introduction opening of the registration facility. The play may first enable easy and resistance-free introduction of the instrument, but second may be as low as possible in order to increase the accuracy. Further, the play may take into account the accuracy of the shape detection by the instrument; if the instrument enables shape detection with millimeter accuracy, the play should also lie in the millimeter range and not substantially above it. A further aspect that may be taken into account when forming the introduction opening of the registration facility are different forms of different shape-detecting instruments. To be able to register differently formed shape-detecting instruments, the introduction opening may have larger dimensions, although this may result in a reduction in the accuracy of the registration of shape-detecting instruments with comparatively small dimensions.

In one embodiment, as a result of the fact that the shape of the shape-detecting instrument is known with the registration, because the shape of the introduction opening of the registration facility may, for example, be assumed to be known, it is not just a single registering position that is registered. Instead, all positions in the introduction opening of the registration facility are likewise registered. In other words, the shape of the shape-detecting instrument is registered. This allows, for example, the calibration or the comparison of the items of shape information generated by the shape-detecting instrument with the coordinate system of the registering facility or the X-ray system.

A development of the present embodiments provides that the shape of the registration facility is a circle or an arc section. This provides that the apparatus is formed such that the shape-detecting instrument is brought into a circular or arcuate shape to enable a precise registration. The circular or arcuate shape represents a mathematically easy-to-describe shape with center, radius, symmetry, and possibly arc angle, and therefore enables a stable mathematical solution to the registration.

One development of the present embodiments provides that the registration facility is formed as a registration channel. The registration channel may have, for example, a round cross-section. Such a channel or such a tube is effectively adjusted to receive an endoscope, laparoscope, catheter, or guide wire. For example, the shape of a channel with a round cross-section is similar to the underlying shape of a blood vessel and is therefore particularly well suited for introducing an instrument for blood vessels, such as guide wire or catheter. Further, a channel (e.g., a channel with a round cross-section) is easy to manufacture and easy to integrate in an apparatus.

A development of the present embodiments provides that the registration facility has an end stop that limits or stops the introduction of the shape-detecting instrument. The end stop may be provided, for example, in an uncomplicated manner in that the introduction space or the introduction opening of the registration facility extends only up to the end stop and ends there. For example, the end stop may be the end of a registration facility configured as a registration channel. In the case of the end stop, it may also any other blocker, stopper, abutment, or limit that makes further introduction of the shape-detecting instrument impossible.

The end stop in the registration facility simplifies handling since the introduction of the shape-detecting instrument is automatically ended and in the correct position (e.g., the registering position). A user therefore does not need to pay attention to attaining the correct position. In addition, an incorrect positioning of the shape-detecting instrument is reliably avoided.

A development of the present embodiments provides that the detecting facility is configured to detect when the shape-detecting instrument touches the end stop. The detecting facility may be configured, for example, as a contact sensor or touch sensor. These may be conventional and available sensors. Contact and touch sensors are uncomplicated to integrate, work reliably, and are inexpensively available.

A development of the present embodiments provides that the registration facility is integrated in a patient table of the X-ray system. Basically, the registration facility may be integrated in any components of the X-ray system. The single condition for this is that the registration facility is arranged in the region of the imaging of the X-ray system. The positioning in the patient table favors simple and precise registration since the patient table is arranged in the region of the imaging of the X-ray system and is thereby situated in the registration region relevant to the imaging. The position of the patient table in the coordinate system of the X-ray system may be known from the outset if a corresponding model of the overall system including patient table and X-ray system is available. Otherwise, the position of the patient table in the coordinate system of the X-ray system may also be registered in advance in that the X-ray system records one or a few X-ray photograph(s) of the patient table. These photographs may be recorded without patients for the purpose of dose reduction. It is to be provided that the known position of the patient table is kept unchanged at least for carrying out the reciprocal registration of the shape-detecting instrument and the X-ray system.

The position of the registration facility in the coordinate system of the X-ray system is to be provided for the registration. If, however, the position of the registration facility within the patient table is known, then, the registration of the patient table also indirectly results in the registration of the registration facility in the coordinate system of the X-ray system. Otherwise, the registration facility may also be registered in the coordinate system of the X-ray system in that the X-ray system records one or a number of X-ray photographs of the registration facility in advance.

The method of the present embodiments includes introducing the shape-detecting instrument into a registration facility until the shape-detecting instrument attains a registering position. The spatial relative position of the registering position in relation to the X-ray system is known. The method also includes detecting when the shape-detecting instrument attains the registering position, and registering the shape-detecting instrument with the X-ray system as soon as attainment of the registering position is detected. Registering takes place by including the known spatial relative position.

The method of the present embodiments has two primary elements, for example, the introduction of the shape-detecting instrument into a registration facility and detecting when the shape-detecting instrument attains the registration position in the registration facility.

The registration facility is an apparatus or apparatus component that brings the instrument into the registering position. The detecting facility is, for example, a device that recognizes when the instrument has attained the registering position, and then generates a signal. The detecting facility may be configured, for example, as a contact sensor or touch sensor, or as a capacitive or optical sensor. These may also be conventional and available sensors that are uncomplicated, reliable, and inexpensive.

The registering position is the position that the instrument is to adopt in the registration facility in order to be correctly registered. The spatial relative position of the registering position in relation to the X-ray system is known. This provides that the spatial relative position may be assumed to be known from the outset for carrying out the registration and does not have to be ascertained or reconstructed specifically for carrying out the registration. The spatial relative position may be predefined in terms of construction, for example, by the patient table. The position of the patient table in relation to the X-ray system may be known in the controller of X-ray system and patient table. The spatial relative position may also have been ascertained in advance by the X-ray system based on X-ray photographs (e.g., have been registered with the X-ray system therefore). So that the relative position of the registering position in relation to the X-ray system is assumed to be known, provides therefore that the ascertainment thereof is not part of the method of the present embodiments. Because it is known from the outset, time, effort, and work steps of the method of the present embodiments are reduced. The registering signal is generated by the detecting facility when the instrument has attained the registering position. A computer-implemented registration method may ascertain the reciprocal registration of instrument and X-ray system in response to the generation of the registering signal.

The registration method may be carried out by the X-ray system or by the shape-detecting instrument because the X-ray system and the shape-detecting instrument each have a coordinate system. The X-ray system includes a control facility, the shape-detecting instrument, and a monitoring facility, and one of the two facilities or both may carry out the registration method. Therefore, the registering signal is transmitted by the detecting facility either to control facility of the X-ray system or to the monitoring facility of the shape-detecting instrument. As soon as the registering signal is received, the registration method is started in the receiving facility.

The known spatial relative position of the registering position in relation to the X-ray system is utilized in the reciprocal registration. Since the spatial relative position is known, in other words, the position of the registering position in the coordinate system of the X-ray system is known. Since the registration takes place when the shape-detecting instrument has attained the registering position, therefore, the position of the shape-detecting instrument in the coordinate system of the X-ray system is also known. Based on the known position in the registering position and the knowledge that the shape-detecting instrument is in the registering position, a registration is thus possible without further items of information being necessary or X-ray photographs having to be made.

To be able to ascertain a spatial registration, knowledge of the position of a plurality of points in the space is necessary. In a two-dimensional coordinate system, the positions of at least two points would be necessary for a registration or in a three-dimensional coordinate system, the positions of at least three points. The term “registering position” does not refer therefore to the position of a single point alone.

One possible embodiment provides ascertaining the length of the registering facility or the registration channel. The measuring points of the shape-detecting instrument on this length starting from the tip of the instrument are ascertained. The number of measuring points depends on the length and also on the spatial resolution of the shape-detecting instrument. The shape of the registering facility is sampled in a corresponding number of points. The two quantities of points (e.g., measuring points) of the shape-detecting instrument and sample points of the registering facility are then mapped onto each other in order to thus ascertain the reciprocal registration.

With the reciprocal registration of the coordinate systems, the positions are used in conjunction with the registering position, which result first in the coordinate system of the shape-detecting instrument and second in the coordinate system of the X-ray system. In other words, these positions are ascertained by the shape-detecting instrument in its coordinate system. As explained above, these positions are known in the coordinate system of the X-ray system. The transformation between the coordinate systems or the reciprocal registration is then ascertained in a known manner based on the known positions of conforming points in the space in the respective coordinate systems.

The present embodiments may reduce the effort and time requirement for the registration of the coordinate systems since no additional X-ray images are to be recorded. The present embodiments may improve the precision of the registration since with the previously known registering position, the exact relative position of the instrument in relation to the X-ray system is known. The present embodiments may reduce the effort since no additional steps, such as recording X-ray images, are necessary, and since the registration method may be automatically triggered based on the registering signal. The present embodiments may reduce the exposure to radiation for patients and medical staff since no additional X-ray images are to be recorded for the purpose of registration.

In a development of the method according to the present embodiments, when introduced into the registration facility, the shape-detecting instrument is brought into a shape similar to the shape of the introduction opening of the registration facility, and registering takes place by including the shape of the introduction opening of the registration facility. As stated above, knowledge of the position of a plurality of points in the space is necessary for a spatial registration. Combined, a large number of points may also be regarded as a shape. A reciprocal registration may be carried out for a shape with less effort and more precisely than for a large number of individual points.

Further, suitable shapes may be simply described as a mathematical model. If the shape is, for example, a circle or an arc section, then a simple mathematical description with center, radius, symmetry, and possibly arc angles is available. A reciprocal registration may be carried out in an uncomplicated and precise manner for shapes that may be simply described as a model.

In a development of the method according to the present embodiments, detecting when the shape-detecting instrument attains the registering position takes place via a detecting facility. The detecting facility is, as explained above, a facility that recognizes when the instrument has attained the registering position, and then generates a signal. The detecting facility may be configured, for example, as a contact sensor or touch sensor, or as a capacitive or optical sensor.

In a development of the method according to the present embodiments, detecting when the shape-detecting instrument attains the registering position takes place via the shape-detecting instrument itself. For example, the shape-detecting instrument may establish whether changes in its shape stop suddenly and abruptly on introduction into the detecting facility. For example, the shape-detecting instrument may be fitted with a sensor system for pressure and establish that when an end stop is attained, it is possible to suddenly and abruptly measure pressure. For example, the shape-detecting instrument may be fitted with a sensor system for an internal rotation and establish that when an end stop is attained, it is possible to suddenly and abruptly measure rotations. For example, the registering facility may be fitted with a particular and characteristic shape feature (e.g., a terminal curve or straight line that may be detected by the shape-detecting instrument).

The items of shape information of the shape-detecting instrument are to be suitably evaluated for this purpose. As a result of this evaluation, when detecting when the shape-detecting instrument attains the registering position, the registering signal is generated. This evaluation may either take place in the monitoring facility of the shape-detecting instrument, for which reason the monitoring facility is to read-in or receive the criteria for recognizing the registering position in advance. The evaluation may also take place in the control facility of the X-ray system or in a registration computer assigned to it. For this reason, the respective system is to read-in or receive in the items of shape information of the shape-detecting instrument as well as the criteria for recognizing the registering position in advance.

### BRIEF DESCRIPTION OF THE DRAWINGS

FIG. 1 shows one embodiment of an X-ray system with patient table and registration facility;

FIG. 2 shows one embodiment of an X-ray system with patient table and registration facility;

FIG. 3 shows one embodiment of a patient table with registration facility and shape-detecting instrument;

FIG. 4 shows one embodiment of a patient table with registration facility, detecting facility, and shape-detecting instrument;

FIG. 5 shows one embodiment of a patient table with registration facility and shape-detecting instrument;

FIG. 6 shows one embodiment of a patient table with registration facility and detecting facility as well as shape-detecting instrument; and

FIG. 7 shows one embodiment of a patient table with registration facility, detecting facility, and shape-detecting instrument.

### DETAILED DESCRIPTION

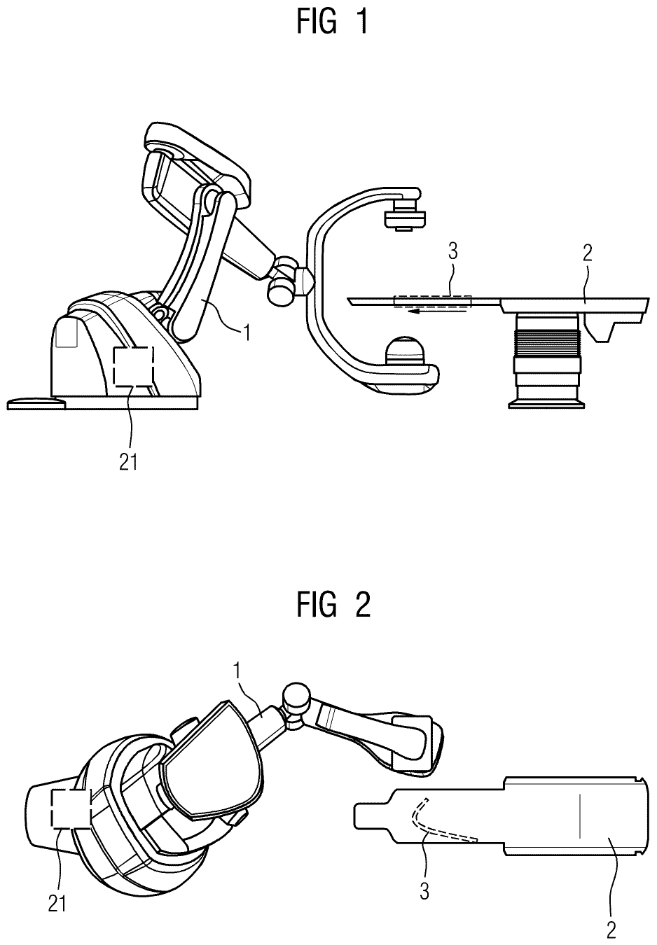

FIG. 1 schematically represents an X-ray system 1 and a patient table 2. Arranged inside the patient table 2 is an introduction opening of a registration facility configured as a registration channel 3. The registration channel 3 is arranged in the portion of the patient table 2 that faces the X-ray system 1.

FIG. 2 schematically represents, from above, the X-ray system 1 with the patient table 2. The registration channel 3 is arranged inside the patient table 2. For the purpose of explanation, the course of the registration channel 3 is drawn so as to be visible, however. The registration channel 3 extends in a curve. As a result, the individual portions of the registration channel 3 are not spatially located in a line, and this enables a more precise registration in the coordinate system of the X-ray system.

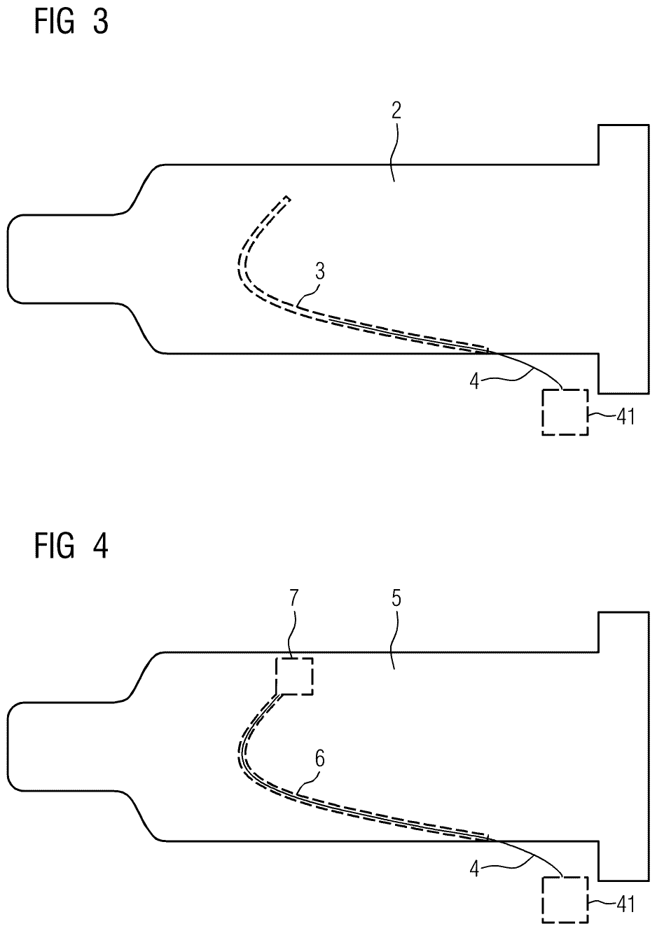

FIG. 3 schematically represents the patient table 2 from above. A shape-detecting instrument 4 is partially introduced into the registration channel 3. The course of the shape-detecting instrument 4 is situated inside the registration channel 3, but for the purpose of explanation, is drawn so as to be visible. The shape-detecting instrument 4 may be inserted up to an end of the registration channel 3. The insertion is stopped by the end of the registration channel 3.

In one embodiment, the items of shape information detected by the shape-detecting instrument 4 describe the course of the registration channel 3 in a coordinate system of the shape-detecting instrument 4. The items of shape information of the shape-detecting instrument 4 also include items of position information in the coordinate system of the shape-detecting instrument 4. The shape and the position or positions of the registration channel 3 in the coordinate system of the X-ray system 1 are known. Either there is a model available of the patient table 2, the X-ray system 1 and the registration channel 3 from the outset for the registration method, so shape and position are known, or otherwise the X-ray system 1 may have recorded one or more X-ray photographs that represent the registration channel 3, and have registered shape and position based on these X-ray photographs.

FIG. 4 schematically represents a patient table 5 from above. A registration channel 6 is arranged inside the patient table 5. However, for the purpose of explanation, the registration channel 6 is drawn so as to be visible. Extending inside the registration channel 6 is a shape-detecting instrument 4 that has been introduced therein. The shape-detecting instrument 4 is introduced up to its end into the registration channel 6. The end of the registration channel 6 is closed and represents an end stop, up to which the shape-detecting instrument 4 is introduced. The end stop stops the shape-detecting instrument 4 and cannot be introduced or advanced further.

In a region of the end stop, there is a detecting facility 7 that detects when the shape-detecting instrument 4 touches the end stop, and then generates a registering signal. The registering signal may be sent to the control facility 21 of the X-ray system or to the monitoring facility 41 of the shape-detecting instrument 4 or to both facilities. The registering signal initiates a reciprocal registration of the coordinate systems of the X-ray system 1 and of the shape-detecting instrument 4. The control facility 21 or the monitoring facility 41 carry out the registration method when the control facility 21 or the monitoring facility 41 receives the registering signal.

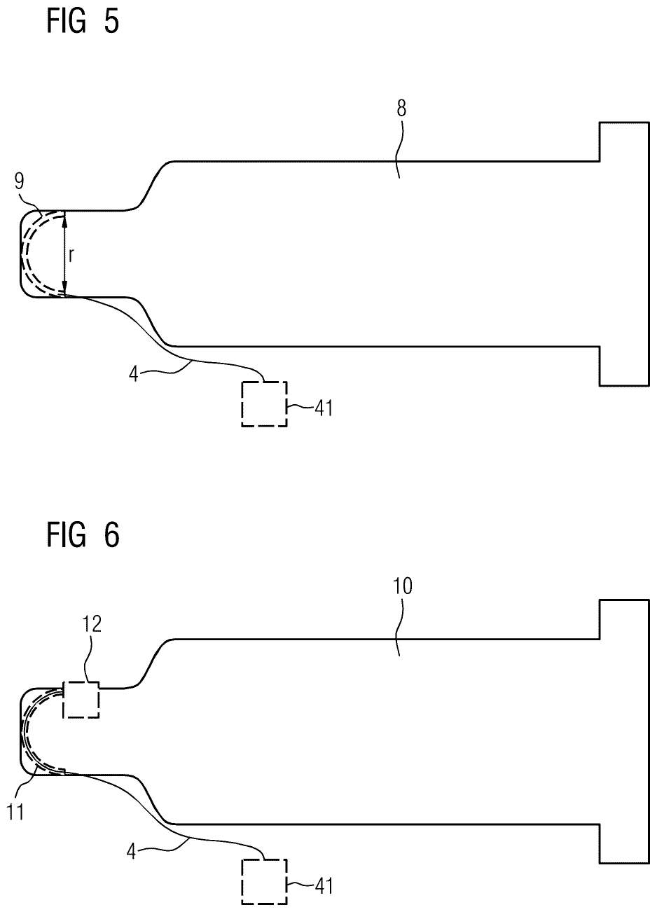

FIG. 5 schematically represents a patient table 8 from above. A registration channel 9 is represented in an end region of the patient table 8. The registration channel 9 is arranged inside the patient table 8 but is drawn so as to be visible for the purpose of explanation. A shape-detecting instrument 4 is inserted into an initial region of the registration channel 9. The shape-detecting instrument 4 may be inserted up to the end of the registration channel 9. The insertion is stopped by the end of the registration channel 9.

The registration channel 9 is formed as an arc section. An arc section is a shape that may be easily described by a mathematical model. The simple mathematical model enables an uncomplicated and precise registration based on its parameters: center, radius, and arc angle.

FIG. 6 schematically represents a patient table 10 with a registration channel 11 from above. A detecting facility 12 is situated in a region of the end stop of the registration channel 11. The detecting facility 12 detects when the shape-detecting instrument 4 touches the end stop, and then generates a registering signal. The registering signal is sent to the control facility 21 of the X-ray system 1 or the monitoring facility of the shape-detecting instrument 4, or to both. As a result, a reciprocal registration of the coordinate systems of the X-ray system 1 and of the shape-detecting instrument 4 is initiated.

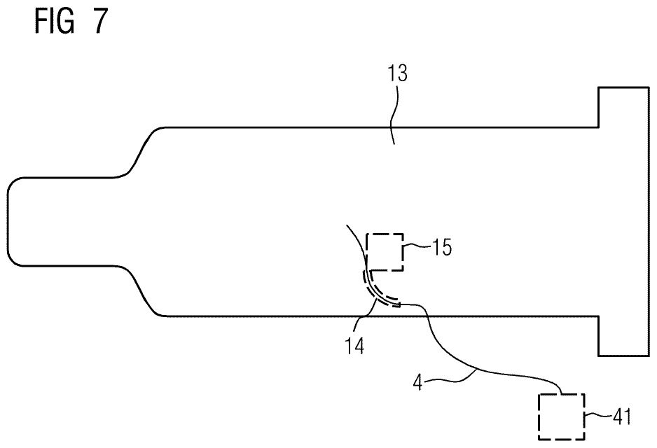

FIG. 7 schematically represents a patient table 13 with a registration channel 14 from above. A detecting facility 15 is situated in an end region of the registration channel 14. The detecting facility 15 detects when the shape-detecting instrument 4 attains the end region, and then generates a registering signal. The registering signal is sent to the control facility 21 of the X-ray system 1, or the monitoring facility of the shape-detecting instrument 4, or to both. As a result, a reciprocal registration of the coordinate systems of the X-ray system 1 and of the shape-detecting instrument 4 is initiated.

The position and shape of the shape-detecting instrument 4 adopted by the shape-detecting instrument 4 when the shape-detecting instrument 4 has attained the end region of the registration channel 14 is used as the basis for the reciprocal registration. This is established by the detecting facility 15. However, the registration channel 14 is open in the end region and may be passed by the shape-detecting instrument 4. The shape-detecting instrument 4 may therefore leave the registration channel 14 again in the end region and be advanced further. The reciprocal registration of the coordinate systems of the X-ray system 1 and of the shape-detecting instrument 4 may therefore be performed intermediate step during the advance of the shape-detecting instrument 4.

In this way, it is possible to perform the reciprocal registration of the coordinate systems of the X-ray system 1 and of the shape-detecting instrument 4 without the shape-detecting instrument 4 then having to be withdrawn again backwards from the registration channel 14. Instead, the shape-detecting instrument 4 may be pushed through further forwards. Following the registration, the site of the intervention may be attained via further advancing of the shape-detecting instrument 4. This simplifies and facilities handling since the shape-detecting instrument 4 does not initially have to be withdrawn backwards and then, conversely, advanced forwards again to the site of the intervention.

In the preceding description, independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An apparatus for reciprocal registration of a shape-detecting instrument and an X-ray system, the apparatus comprising:
a registration facility configured such that the shape-detecting instrument, until attaining a registering position, is introducible into the registration facility, wherein a spatial relative position of the registering position is keepable at least temporarily unchanged in relation to the X-ray system; and
a detecting facility configured to detect when the shape-detecting instrument attains the registering position, wherein a registering signal is generatable by the detecting facility when the detecting facility detects that the shape-detecting instrument attains the registering position.

2. The apparatus of claim 1, wherein the registration facility is configured such that when introduced into the registration facility, the shape-detecting instrument is deformed into a shape correspond to a shape of an introduction opening of the registration facility.

3. The apparatus of claim 2, wherein the shape of the introduction opening of the registration facility corresponds to a circle or an arc section.

4. The apparatus of claim 2, wherein the introduction opening of the registration facility is configured as a registration channel into which the shape-detecting instrument is introducible.

5. The apparatus of claim 1, wherein the registration facility has an end stop configured to stop further insertion of the shape-detecting instrument when the registering position is attained.

6. The apparatus of claim 5, wherein the detecting facility is configured to detect when the shape-detecting instrument touches the end stop.

7. The apparatus of claim 1, wherein the registration facility is integrated in a patient table of the X-ray system.

8. An X-ray system comprising:
an apparatus for reciprocal registration of a shape-detecting instrument and an X-ray system, the apparatus comprising:
a registration facility configured such that the shape-detecting instrument, until attaining a registering position, is introducible into the registration facility, wherein a spatial relative position of the registering position is keepable at least temporarily unchanged in relation to the X-ray system; and
a detecting facility configured to detect when the shape-detecting instrument attains the registering position, wherein a registering signal is generatable by the detecting facility when the detecting facility detects that the shape-detecting instrument attains the registering position.

9. A method for reciprocal registering of a shape-detecting instrument and an X-ray system, the method comprising:
introducing the shape-detecting instrument into a registration facility until a registering position is attained, wherein a spatial relative position of the registering position in relation to the X-ray system is known;
detecting when the shape-detecting instrument attains the registering position; and
registering the shape-detecting instrument with the X-ray system as soon as attainment of the registering position is detected,
wherein the registering comprises registering including the known spatial relative position.

10. The method of claim 9, wherein the registration facility is configured such that when introduced into the registration facility, the shape-detecting instrument is deformed into a shape corresponding to a shape of an introduction opening of the registration facility, and
wherein the registering further comprises registering including the known shape of the introduction opening.

11. The method of claim 9, wherein detecting when the shape-detecting instrument attains the registering position takes place via a detecting facility.

12. The method of claim 9, wherein the shape-detecting instrument detects when the shape-detecting instrument attains the registering position.
